(19) United States
(12) Patent Application Publication
(10) Pub. No.: US 2011/0230922 A1
(43) Pub. Date: Sep. 22, 2011

(54) Device And Method For Peri-Hisian Pacing And/Or Simultaneous Bi-Ventricular or Tri-Ventricular Pacing For Cardiac Resynchronization

(76) Inventor: Robert S. Fishel, Delray Beach, FL (US)

(21) Appl. No.: 13/050,566

(22) Filed: Mar. 17, 2011

Related U.S. Application Data
(60) Provisional application No. 61/314,731, filed on Mar. 17, 2010.

(51) Int. Cl. A61N 1/365; A61N 1/362; A61N 1/39

(52) U.S. Cl. 607/4; 607/36; 607/17

(57) ABSTRACT

The present invention provides a pacing device and method that allows for preferential right atrial, at or near the His bundle, and ventricular pacing, either individually or in combination, including tri-ventricular pacing. The pacing device includes a power source and one or more logic circuits allowing for programmable delivery of pacing to any combination of the right atria, the para-Hisian region, and the right and/or the left ventricles. The pacing device allows the user to select which site(s) to pace as well at the appropriate relative timing of pacing impulse delivery to any of these three previously mentioned sites. The device is constructed and arranged to be combined with an atrial sensing/pacing electrode to allow for atrio-ventricular sequential tri-ventricular pacing or variants of such. Also, a defibrillator lead can be incorporated into the device to allow for protection from ventricular arrhythmias and sudden death.

### CROSS REFERENCE TO RELATED APPLICATIONS

This Application claims priority under 35 U.S.C. §119(e) to U.S. Provisional Application 61/314,731, filed on Mar. 17, 2010, the contents of which are herein incorporated by reference.

### FIELD OF THE INVENTION

The instant invention relates to cardiac pacing devices and methods of cardiac pacing, and more particularly to a pacing device and method that allows for preferential His/para-Hisian pacing (PHP), right atrial pacing, and ventricle pacing, individually or in combination of the three sites, thereby providing improved cardiac resynchronization therapy.

### BACKGROUND OF THE INVENTION

The heart is a vital aspect of human physiology responsible for transporting and exchanging substances between the environment and the cells that make up the various tissues and organs. The main function of the heart is to pump blood through the circulatory system using coordinated, rhythmic contractions. Such contractions are based on a complex series of electrocardio and mechanocardio events. Briefly, during periods of contractions, the heart muscles pump blood out through the arteries. During periods of relaxation, the heart fills with deoxygenated blood in the right ventricles, and oxygenated blood in the left ventricles. The main driving forces for contraction and relaxation rhythms are the electrocardial events. The sinoatrial node (SA node), often called the natural pacemaker, is part of the heart's natural conduction system and is made of specialized cells that are capable of generating electrical signals. Electrical events are generated in the SA node, eventually spreading to the rest of the heart via nodal tissue pathways which coordinate the events of the cardiac cycle. The electrical signals generated through the pathway cause the heart muscles to squeeze and release in a coordinated, rhythmic sequence that result in drawing blood into the heart chambers and forcing blood out of the chambers.

Despite advances in the understanding and treatment of cardiac diseases, heart failure remains a major issue and cause of concern for physicians. It is estimated that heart failure affects millions of individuals in the United States, and tens of millions of people worldwide. Those suffering from congestive heart failure (CHF) often have a poor quality of life and have high mortality rates. Early pacing methods used single chamber pacing with leads placed in the right atrium, the right ventricle, or the left ventricle. Such pacing methods, however, failed to provide complete synchronization of the chambers of the heart and the hemodynamic output was not always sufficient. For example, pacing the right atrium only was not effective for disease states in which cardiac signals were blocked at a position distal to the right atrium. Pacing from the right ventricle can create asynchronous contraction of the left and right ventricles. Such contractions result in inefficient mechanical contraction and reduced hemodynamic performance. Moreover, long term right ventricular pacing has been found to be associated with an increased risk of developing and/or worsening heart failure.

One promising method which has gained a lot of attention for treating arrhythmias over the last ten years is cardiac resynchronization therapy. Cardiac resynchronization therapy (CRT) pacing is a technique allowing for pacing of both the right ventricle (RV) and the left ventricle (LV) simultaneously to allow for synchronous contraction of both ventricles during a cardiac cycle. In patients suffering from dysynchronous heart functions, such as those who acquire a left bundle branch block, the native conduction system of the left ventricle no longer conducts heart beats delivered to the AV node from the atria. In this situation, the left ventricle follows the right ventricle in its contraction, with the left ventricle contraction occurring via slow cell to cell conduction as opposed to the much faster conduction present in the setting of an intact left bundle branch. This situation causes cardiac dysynchrony and there is no longer a coordinated contraction between the right ventricle and left ventricle during any given heart beat. In cardiac resynchronization therapy, an additional left ventricle pacing lead is delivered to a region of the left ventricle either via an epicardial or endocardial approach and left ventricle pacing is provided at the same time as right ventricle pacing, allowing for synchronous contraction of both ventricles. Cardiac resynchronization therapy pacing will typically improve a patients' ejection fraction and decrease His symptoms of congestive heart failure if cardiac resynchronization therapy is performed correctly. A significant number of patients fail to demonstrate any clinical benefit to CRT using biventricular pacing which may be attributable to the unsuccessful implantation and placement of the pacing leads.

Therefore, what is needed in the art is a pacing device and method that overcomes the shortcomings of the art and results in synchronization of the heart chambers which is the same as, or similar to, the natural, non-diseased state synchronization.

### DESCRIPTION OF THE PRIOR ART

One of the main goals of cardiac stimulation methods is the restoration of the physiological electrocardio sequence of the heart in order to maintain an adequate heart rhythm. Cardiac resynchronization using biventricular pacing has been an established treatment option for patients in which single chamber pacing is not providing relief or is not a viable option. While many patients find success in such a therapy, a significant number of patients fail to demonstrate clinical benefits to the therapy. An alternative to biventricular pacing is direct pacing of the His bundle. Electrical stimulation of the His Bundle was used in the 1970s for diagnostic purposes, see Occhetta et al., Permanent direct His bundle pacing a novel approach to cardiac pacing in patients with normal His-Purkinje activation. Circulation 2000; 101; 869-877. In the 1990s, several researches developed His bundle stimulation techniques for therapeutic purposes. Deshmukh et al. published a report in 2000 describing direct His bundle pacing to patients suffering from chronic atrial fibrillation, dilated cardiomyopahty, NYHA functional class III to IV, and spontaneous narrow QRS complexes, see Permanent direct His bundle pacing novel approach to cardiac pacing in patients with Normal His-Purkinje activation. Circulation 2000. In 2006, Occhetta et al. published a study regarding the feasibility and safety induced by permanent para-Hisian pacing in patients with chronic atrial fibrillation and narrow QRS who underwent atrioventricular node ablation, see Prevention of ventricular desynchronization by permanent para-Hisian pacing after atrioventricular node ablation in chronic atrial fibrillation. J Am Coll Cardiol, 2006: 47:1938-1945.

U.S. Pat. No. 7,647,124 describes a catheter for delivering and implanting an electrical lead to a right atrium of a heart in close proximity to a His bundle. The catheter includes proximal and distal portions. The distal portion is described as being hook-shaped, generally planar, and terminating in a distal tip. The distal portion may also have first and second segments, where the second segment is distal of the first. The second segment is described as having curves through an arc of about 100 to 160 degrees to orient the distal tip generally perpendicular to a His bundle when the catheter is implanted. The first segment includes a curvature that springs and orients the second portion towards the His bundle when the catheter is implanted.

United States Patent Application 2010/0228330 describes an implantable medical lead for implantation within a right ventricle of a heart and powered by an implantable pulse generator. The lead is described as including a lead body having a proximal end configured to couple to the generator, a distal end, an electrode at the distal end, and a distal portion extending proximally from the distal end. When the distal portion is in a non-deflected state, the distal portion biases to assume a configuration including first, second and third generally straight segments and first and second bends. The first segment is proximal of the distal end. The second segment is proximal of the first segment. The third segment is proximal of the second segment. The first bend is positioned between the first and second segments. The second bend is positioned between the second and third segments. When the distal portion is implanted in the right ventricle, the configuration is at least partially the cause of the electrode being at least one of: positioned against the right ventricle septum; positioned in the outflow tract of the right ventricle; positioned for Hisian pacing and positioned for para-Hisian pacing.

United States Patent Application 2009/0259272 describes a system for therapeutically stimulating a His bundle. The system is described as having an implantable pulse generator and a multi-polar medical electrical lead. The generator is configured for subcutaneous implantation and to generate a pacing stimulus. The lead includes a connector assembly, a flexible tubular body, a distal tip assembly and coil conductors. The body extends intravascularly from the generator to a location proximate the His bundle and includes a proximal end, a distal end, and a longitudinal lumen. The tip assembly includes an electrode, a fixation helix, and a shank portion. The helix extends to a location proximate the His bundle and is operable as an electrically isolated electrode. The shank portion extends within the lumen and includes a receptacle for receiving a stylet tip. The conductors extend longitudinally through the lumen and are coupled to the electrode and the helix. One or both of the conductors defines a stylet lumen.

### SUMMARY OF THE INVENTION

Normal cardiac physiology requires participation of the specialized conduction system of the ventricles to allow for simultaneous and rapid depolarization of both the right and left ventricles. This allows for a synchronous and coordinated contraction of both ventricles, coordinated activation of the papillary muscles, preservation of atrio-ventricular valve function and maximization of cardiac output. In disease states, abnormal function of either the bundle of His, the AV node, the right bundle branch or the left bundle branch can prevent simultaneous activation of both ventricles via the normal His-Purkinje system. Such a disease state often results in discordant contraction of the left and right ventricles, decreased cardiac output, as well as numerous other impairments to normal cardiac physiology.

Bi-ventricular pacing attempts to resolve some of these issues by delivering pacing to both the right and left ventricles. This technique however is imperfect as activation of the normal His-purkinje system generally does not occur with biventricular pacing resulting in a cardiac contraction which, while improved versus right bundle branch only conduction, is still non-physiologic. Recent clinical evidence demonstrates that para-Hisian pacing (PHP) at or near the vicinity of the bundle of His and/or compact AV nodal region can result in biventricular native bundle branch conduction despite the presence of a left bundle branch block at baseline. This phenomenon is thought to be the result of recruitment of actively conducting left bundle branch tissue below the point of block. The finding that PHP allows for bilateral bundle branch activation suggests that this technique might prove to be of better physiologic benefit then conventional biventricular pacing. PHP alone however has potential problems including inappropriate sensing of atrial potentials and possibly inconsistent or not entirely reliable long term ventricular capture.

The present invention provides a pacing device and method that allows for preferential right atrial, His bundle, right ventricular pacing, left ventricular pacing, either individually or in combination, including tri-ventricular pacing. The pacing device allows a user to select which site(s) to pace as well as at the appropriate relative timing of pacing impulse delivery to any of the three previously mentioned sites. The device therefore provides optimizing resynchronization by choosing which combination of electrodes, i.e., right ventricle to left ventricle, His alone, His to right ventricle to left ventricle, left ventricle to His, and right ventricle to His, results in improvement to the patient's condition. The pacing device further provides for resynchronization redundancy in the event that one of the multiple electrodes in the ventricles (His, right ventricle, left ventricle) fails to either pace or sense properly. The pacing device includes an enclosed battery and one or more logic circuits allowing for programmable delivery of pacing to any combination of the right atria, the para-Hisian region, and the right and/or the left ventricles. The device is constructed and arranged to be combined with an atrial sensing/pacing electrode to allow for atrio-ventricular sequential multi-site ventricular pacing or variants of such. Also, an automatic defibrillator can be incorporated into the device to allow for protection from ventricular arrhythmias and sudden death. This device differs from existing devices which do not have the ability to deliver pacing to all three, or a combination of these three sites in the heart. A port to allow for atrial sensing and pacing and atrio-ventricular sequential pacing may also be incorporated into this device. In addition, one may incorporate ports to deliver high voltage shocks to the ventricle to treat life threatening ventricular arrhythmias.

The device can further be adapted for diagnostic purposes. Should atrial potentials be sensed at the point of PHP, the timing sequence of these potentials in reference to the potentials recorded from the electrode placed in the high right atria can be used to help discriminate life-threatening ventricular arrhythmias from generally non life-threatening supra-ventricular arrhythmias. The sequence of timing of the PHP ventricular potentials and those recorded from the right ventricular and left ventricular electrodes could also be used for diagnostic discrimination of arrhythmia types.

Accordingly, it is an objective of the instant invention to provide a pacing device that allows for pacing of the right atrium, the bundle of His, and one or both ventricles.

It is a further objective of the instant invention to provide a pacing device that allows the user the ability to select which site(s) to pace as well as at the appropriate relative timing of pacing impulse delivery to any of the right atrium, the bundle of His, and one or more ventricle sites.

It is yet another objective of the instant invention to provide a method of resynchronizing the electrical signals of the heart by pacing the right atrium, the bundle of His, and one or more ventricular sites.

It is a still further objective of the invention to provide a method of resynchronizing the electrical signals of the heart which allows the user the ability to select which site(s) to pace as well as at the appropriate relative timing of pacing impulse delivery to any of the right atrium, the bundle of His, and one or more ventricular sites.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a schematic view of the pacing device in accordance with the instant invention;

FIG. 2 is a functional block diagram of the pacing device in accordance with the instant invention.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

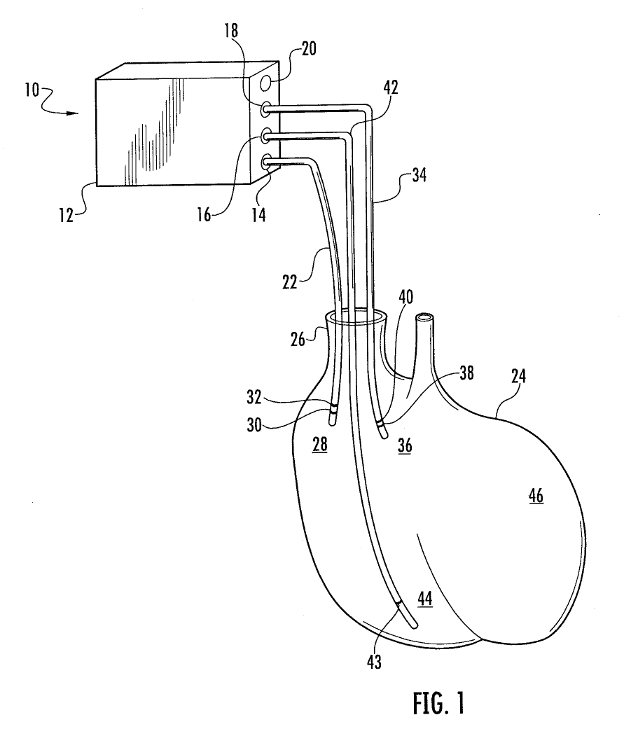

FIG. 1 is an illustrative example of a pacing device for providing His/para-Hisian pacing and/or simultaneous multi-ventricular pacing for cardiac resynchronization. The pacing device 10 contains a pacing device body 12. The pacing body 12 contains a plurality of ports 14, 16, 18, and 20 which are constructed and arranged to couple the main body 12 to various pacing and/or sensing leads. For example, a first lead 22, which is coupled to the port 14, is inserted into the heart 24 through the vena cava 26 and placed within the right atrium 28. The lead contains a pacing electrode 30 for providing a pacing event. This lead will also allow for sensing of intrinsic cardiac events, through, for example, the sensing electrode 32 and/or sensing circuitry. The sensing electrode relays the heart's electrical information back to the sensing circuitry and or the main control unit. A second pacing lead 34 couples to the main pacing device body 12 through port 18. The second pacing lead is inserted into the heart 24 and is placed at or near the His bundle 36. The second pacing lead 34 also contains an electrode 38 for delivering a pacing event to at or near the bundle of His for providing His pacing or para-Hisian pacing.

The second pacing lead 34 can be placed in a position distal in the conduction system chain to the compact atrio-ventricular node and proximate to the bundle of His. The lead will preferably be placed in a region of the His-bundle such that the lead is distal to the point of block for the patient's respective either left or right bundle branch block. Pacing from this lead, if placed correctly, will thereby allow for capture of both the native left and right bundle branches. A paced complex from this electrode will appear on surface electrocardiography as to have a narrow QRS morphology similar to that seen in a patient without conduction system disease or a left/right bundle branch block. The second lead will contain a sensing electrode 40 and/or sensing circuitry for sensing of Hisian and near field atrial and ventricular spontaneous cardiac events.

A third lead 42, coupled to port 16 and having a pacing electrode 43, is placed in the right ventricle 44. The third lead may also contain a sensing electrode. The port 20, or additional ports (not illustrated), may optionally contain additional leads for pacing other parts of the heart including pacing in the coronary sinus branches. For example, one or more additional leads may be used to pace the left ventricle as commonly used in biventricular pacing (i.e. one lead through the coronary sinus vein) or triventricular pacing (pacing through the coronary sinus, the right ventricle and the His-bundle or pacing multiple sites in the coronary sinus or right ventricle and His bundle). The leads may also be inserted into the coronary sinus (not illustrated) of the heart in order to pace the left ventricle 46 only with back-up pacing of the right ventricle and Hisian sites. The pacing device 10 may also include an automatic defibrillator device to allow for protection from life threatening ventricular arrhythmias, atrial arrhythmias and sudden death. Additional ports could be added to provide for high voltage shocks to restore a normal or paced rhythm should an abnormal rhythm or threat to life be sensed by the device.

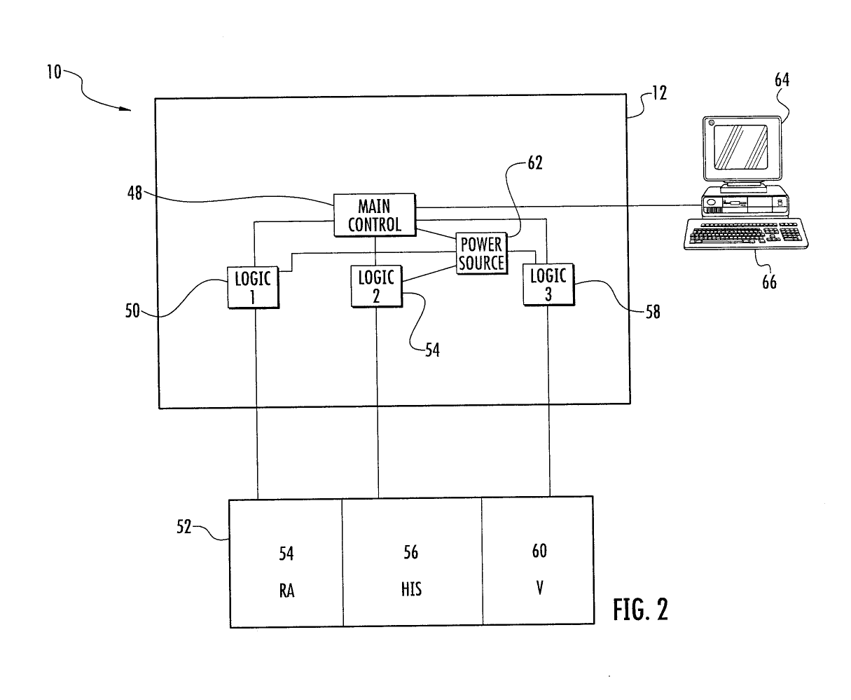

Referring to FIG. 2, a functional block diagram of the pacing device is illustrated, including the interfaces between various aspects of the pacing device 10. The pacing device body 12 contains a control unit 48. The control unit 48 may be a processor with memory, such as a microcomputer, that receives, stores, and sends out information that can be used by other parts of the device to perform various functions. The control unit 48, therefore, is a central aspect of the pacing device 10, providing programmable delivery of pacing to various sites in the heart and/or at programmed times. Coupled to the control unit 48 is a plurality of logic circuitry, each capable of delivering pacing to the various places in the heart 52, as well as cardiac sensing capability. A first logic circuitry 50 provides the capability of delivering programmable pacing to and/or sensing from the right atrium 54 of the heart 52. A second logic circuitry 54 provides the capability of delivering programmable pacing to and/or sensing from a second area of the heart, such as the bundle of His 56. A third logic circuitry 58 provides the capability of delivering programmable pacing to a third area of the heart, such as the ventricles 60, including the left ventricle, the right ventricle, or combinations thereof. The control unit 48, each of the circuit logics, and the generation of pacing events is powered by a power source, illustrated herein as a battery 62.

Coupled to the main control unit 48 is a display unit, illustrated herein as a monitor 64. The monitor 64 can display all the information that the control unit 48 records and can be used by the user to obtain various types of information, such as a history of the electrical activity of the heart or the history of the pacing activity associated with each of the logic circuitry. The monitor may further be used by the user to program the control unit 48 to perform various functions or modifications to the previously programmed functionality. In this manner, both the patient and the patient's cardiologist can use the monitor to observe the hearts electrical and mechanical dynamics and make changes when needed. The monitor 64 may include a data impute device, such as a key board 66 and mouse (not illustrated), buttons or roller pad, or may use electronic visual display technology, such as touch screen technology, that detects the presence and location within a display area, thereby allowing the user the ability to interact with the display unit directly. The monitor 64 may be permanently coupled to the main pacing device body 12 through a cable. Alternatively, the display device may be removeably coupled to the main body 12 through devices that provide communication between the monitor 64 and the main control unit 48, such as cables and Universal Serial Bus (USB) connections. In an alternative embodiment, the main pacing device body 12 and the monitor 64 may be constructed and arranged to be capable of sending and receiving short-range wireless radio waves, such as through the use of blue tooth technology, for providing wireless communications between the two devices.

According to one illustrative implementation, the pacing device 10 is implanted into a patient using surgical procedures known to one of skill in the art. Preferably, the pacing device 10 contains pacing leads which are placed within the right atrium, at or near the His bundle, and within the right ventricle for directly stimulating the normal physiological electrical conduction system of the heart, thereby eliciting a synchronous electrical conduction sequence that is the same as, or similar to, the electrical conduction sequence of the non-diseased heart. The control unit 48 can be programmed to provide pacing to each of the areas of the heart independently or in combination. The pacing event can be programmed to pace continually (asynchronous/fixed rate mode) or can be programmed to pace when needed (synchronous/demand mode). Accordingly, the device 10 contains sensing/pacing electrodes or may include independent atrial sensing electrodes. Which sites paced, as well as the relative timing of the pacing impulse delivery to any of the pacing sites, is controlled by the control unit 48. Depending on the individual physiology of the patient, the device can be programmed to various pacing and timing modes. For example, the device can be programmed to deliver pacing in the ventricular and Hisian channels after an atrial sensed event. The time interval for this delivery of pacing to these channels can be programmed by the physician overseeing the patient based on the patient's individual physiology. Furthermore, which chamber in the ventricles (the right ventricle, the left ventricle, the bundle of His or additional sites on the right/left ventricle) are paced can be determined by the physician to best suit the individual needs of a given patient. If multi-site pacing is needed in the ventricles and bundle of His, then the relative timing of delivery of this pacing, either simultaneously or via individual pacing channels, after an atrial sensed event, can be programmed to best suit the individual patients physiology.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A pacing device for pacing a plurality of regions of the heart comprising: a main pacing device body, said main body housing a power source, a control unit, a plurality of logic circuitry coupled to said control unit, a plurality of ports, said plurality of ports constructed and arranged to receive at least one pacing lead constructed and arranged to pace the right atrium of the heart and at least one pacing lead constructed and arranged to pace at, or near the His bundle region of the heart, wherein pacing of said right atrium and said His bundle region results in synchronization of the heart chambers.

2. The pacing device according to claim 2 further including an additional pacing lead for pacing the right ventricle, said right ventricle pacing lead coupled to said control unit.

3. The pacing device according to claim 2 wherein said atrial pacing lead contains sensing logic for obtaining electrocardial information, said information being used to pace said device.

4. The pacing device according to claim 2 further including a sensing lead, said sensing lead constructed and arranged for obtaining electrocardial information and relaying said information to said sensing circuitry, said information being used to pace said device.

5. The pacing device according to claim 1 further including an additional pacing lead to pace the left ventricle, said left ventricle pacing lead coupled to said main control unit.

6. The pacing device according to claim 1 further including a defibrillator lead.

7. The pacing device according to claim 1 further including a monitor coupled to said control unit.

8. The pacing device according to claim 7 wherein said monitor allows a user to select which site, or combinations of sites, to pace.

9. The pacing device according to claim 7 wherein said monitor allows a user to select the appropriate relative timing of pacing impulse delivery to any of said sites containing said leads.

10. The pacing device according to claim 7 wherein said monitor is wirelessly coupled to said monitor device.

11. The pacing device according to claim 1 wherein said main control unit is programmed to select the sites of pacing.

12. The pacing device according to claim 1 wherein said main control unit is programmed to select the appropriate relative timing of pacing impulse delivery to each of said leads.

13. A method of pacing a plurality of regions of the heart comprising the steps of:
providing to a user a pacing device for pacing a plurality of regions of the heart, said pacing device comprising: a main pacing device body, said main body housing a power source, a control unit, a plurality of logic circuitry coupled to said control unit, a plurality of ports, said plurality of ports constructed and arranged to receive at least one pacing lead constructed and arranged to pace the right atrium of the heart and at least one pacing lead pacing constructed and arranged to pace at, or near the His bundle region of the heart, wherein pacing of said right atrium and said His bundle region results in synchronization of the heart chambers;
coupling said right atrium lead to said control unit of said pacing device and placing said right atrium lead into a region of the right atrium of the heart;
coupling said His bundle lead to said control unit of said pacing device and placing said His bundle lead at or near the vicinity of the bundle of His;
coupling said ventricle lead to said control unit of said pacing device and placing said ventricle lead into the right ventricle;
programming said control unit to pace the right atrium, at or near the His bundle, and the right ventricle at the appropriate relative time; and
providing a pacing event to one or more areas of the heart.

14. The method of pacing a plurality of regions of the heart according to claim 13 wherein said ventricle lead is inserted into a vein of the coronary sinus for pacing the left ventricle.

15. The method of pacing a plurality of regions of the heart according to claim 13 further including a left ventricle lead coupled to said main control unit, said left ventricle lead placed within a vein of the coronary sinus to provide left ventricle pacing.

16. The method of pacing a plurality of regions of the heart according to claim 15 wherein said control unit is programmed to pace the left ventricle at the appropriate relative time.

17. The method of pacing a plurality of regions of the heart according to claim 14 wherein said ventricle lead includes at least two leads inserted into the veins of the coronary sinus for pacing the left ventricle.

18. The method of pacing a plurality of regions of the heart according to claim 17 wherein said control unit is programmed to pace each of the leads to the left ventricle at the appropriate relative time.
